(19) United States
(12) Patent Application Publication
(10) Pub. No.: US 2024/0116990 A1
(43) Pub. Date: Apr. 11, 2024

(54) Compositions and Methods of Treatment Using CRRL 191

(71) Applicant: Mayo Foundation for Medical Education and Research, Rochester, MN (US)

(72) Inventors: John C. BURNETT, Jr., Rochester, MN (US); Shuchong PAN, Rochester, MN (US); Sasantha Jeson SANGARALINGHAM, Rochester, MN (US); Ye ZHENG, Rochester, MN (US)

(73) Assignee: Mayo Foundation for Medical Education and Research, Rochester, MN (US)

(21) Appl. No.: 18/467,797

(22) Filed: Sep. 15, 2023

Related U.S. Application Data
(60) Provisional application No. 63/407,798, filed on Sep. 19, 2022.

(51) Int. Cl. C07K 14/00; A61K 31/341; A61K 45/06; A61P 9/12

(52) U.S. Cl. CPC C07K 14/001; A61K 31/341; A61K 45/06; A61P 9/12; A61K 38/00

(57) ABSTRACT

This document provides methods and materials related to selected engineered analogs of atrial natriuretic peptides. For example, this document provides compositions that contain one or more selected engineered analogs of atrial natriuretic peptides provided herein and that have the ability to treat, prevent, or alleviate the symptoms of cardiovascular, cardiorenal, and metabolic disease.

### CROSS-REFERENCE TO RELATED APPLICATION

This application claims priority to U.S. Provisional Application Ser. No. 63/407,798 filed Sep. 19, 2022, the entirety of which is incorporated by reference herein.

### STATEMENT REGARDING FEDERALLY SPONSORED RESEARCH

This invention was made with government support under HL136340 awarded by the National Institutes of Health. The government has certain rights in the invention.

### INCORPORATION OF SEQUENCE LISTING

This application contains a sequence listing, submitted herewith electronically, containing the file named “P35213US01_Seq_Listing.xml” which is 8,192 bytes in size and was created on Sep. 14, 2023, and which is herein incorporated by reference in its entirety.

### TECHNICAL FIELD

This document provides methods and materials related to selected engineered analogs of atrial natriuretic peptides (ANPs). For example, this document provides compositions that contain one or more selected engineered analogs of atrial natriuretic peptides (ANPs) provided herein and that have the ability to treat cardiovascular (e.g., hypertension, heart failure, cardiomyopathies, valvular disease, myocardial infarction), cardiorenal (e.g., acute kidney injury, chronic kidney disease, polycystic kidney disease, chemotherapeutic-induced cardiac and renal dysfunction), and metabolic diseases (e.g., diabetes, obesity, metabolic syndrome, hyperlipidemia, insulin resistance, primary aldosteronism, NASH) within a mammal (e.g., a human).

### BACKGROUND INFORMATION

Natriuretic polypeptides are polypeptides that can cause natriuresis—increased sodium excretion in the urine. Natriuretic polypeptides can be produced in the brain, heart, kidney, and/or vascular tissue. The natriuretic polypeptide family in humans includes the cardiac hormones atrial natriuretic peptide (ANP), B-type natriuretic peptide (BNP), C-type natriuretic peptide (CNP), Dendroaspis natriuretic peptide (DNP) isolated from the green mamba snake, and urodilatin (URO). Natriuretic polypeptides bind to two well-characterized guanylyl cyclase receptors, GC-A and GC-B (also called natriuretic peptide receptors (NPR-A and NPR-B)). GC-A is the primary receptor for ANP, BNP, DNP, and URO; and GC-B is the primary receptor for CNP). Once bound, the receptors in turn catalyze the conversion of guanosine triphosphate (GTP) to the second messenger cyclic 3′5′ guanosine monophosphate (cGMP) (Kuhn, Circ. Res., 93:700-709 (2003); Tawaragi et al., Biochem. Biophys. Res. Comm., 175:645-651 (1991); and Komatsu et al., Endocrinol., 129:1104-1106 (1991)). The downstream effects of GC-A receptor activation include natriuresis, arterial vasodilation, renin and aldosterone suppression, anti-apoptosis, anti-hypertrophy, lusitropy, vascular regeneration, lipolysis, and browning of white adipocytes. The downstream effects of GC-B receptor activation include anti-fibrosis, anti-inflammation, vascular regeneration, microcirculatory dilation, and lusitropy.

Previous disclosures, e.g., International Patent Application No. PCT/US2017/060808, provided analogs of alternatively-spliced atrial natriuretic peptides (MANP) consisting of the 28 amino acids of native ANP and a 12 amino acid extension on the C-terminus. MANP, compared to ANP, is more resistant to degradation, has enhanced binding to GC-A receptor, causes increased and more sustained sodium excretion, causes more sustained suppression of aldosterone, and leads to greater and more sustained reduction in blood pressure.

### SUMMARY

The present disclosure provides design and validation of a next-generation class of novel GC-A activators, with improved binding to GC-A receptors, greater activation of cGMP, increased resistance to neprilysin degradation, and greater efficacy than ANP in vivo in lowering blood pressure.

The present disclosure provides, and includes, methods and materials related to selected engineered analogs of atrial natriuretic peptides (ANPs). For example, the present disclosure provides the engineered polypeptides set forth in Table 1, that comprise, consist essentially of, or consist of the amino acid sequence set forth in any one of SEQ ID NOs: 3-7.

In an aspect, a selected engineered analog of ANP provided herein is a polypeptide comprising the amino acid sequence set forth in any one of SEQ ID NOs: 3-7. In an aspect, a selected engineered analog of ANP provided herein is a polypeptide comprising the amino acid sequence set forth in any one of SEQ ID NOs: 3-7, wherein the polypeptide further comprises one, two, or three amino acid substitutions, additions, or deletions. In an aspect, a selected engineered analog of ANP provided herein is a polypeptide comprising a sequence having at least 90% homology with any one of SEQ ID NO: 3-7. In an aspect, the polypeptide sequence has at least 95% homology with any one of SEQ ID NO: 3-7. In an aspect, the polypeptide sequence has at least 97% homology with any one of SEQ ID NO: 3-7. In an aspect, a selected engineered analog of ANP provided herein is a polypeptide comprising the amino acid sequence set forth in amino acids 1 to 8 of SEQ ID NO 7.

The present disclosure provides, and includes, substantially pure polypeptides related to selected engineered analogs of ANPs. In an aspect, a selected engineered analog of ANP provided herein is a substantially pure polypeptide comprising an amino acid sequence set forth in any one of SEQ ID NOs: 3-7. In an aspect, a selected engineered analog of ANP provided herein is a substantially pure polypeptide comprising an amino acid sequence having at least 90% homology with any one of SEQ ID NOs: 3-7. In an aspect, the substantially pure polypeptide has a sequence that has at least 95% homology with any one of SEQ ID NO: 3-7. In an aspect, the substantially pure polypeptide has a sequence that has at least 97% homology with any one of SEQ ID NO: 3-7. In an aspect, a selected engineered analog of ANP provided herein is a substantially pure polypeptide comprising the amino acid sequence set forth in any one of SEQ ID NOs: 3-7, wherein the polypeptide comprises one, two, or three amino acid substitutions, additions, or deletions. In an aspect, a selected engineered analog of ANP provided herein is a substantially pure polypeptide comprising the amino acid sequence set forth in amino acids 1 to 8 of SEQ ID NO 7.

The present disclosure also provides compositions of selected engineered analogs of ANPs provided herein. In an aspect, a composition provided herein comprises a polypeptide comprising the amino acid sequence set forth in any one of SEQ ID NOs: 3-7. In an aspect, a composition provided herein comprises a polypeptide comprising the amino acid sequence set forth in any one of SEQ ID NOs: 3-7, wherein the polypeptide further comprises one, two, or three amino acid substitutions, additions, or deletions. In an aspect, a composition provided herein comprises at least two polypeptides, wherein each of said at least two polypeptides is a polypeptide comprising the amino acid sequence set forth in any one of SEQ ID NOs: 3-7. In an aspect, a composition provided herein comprises the amino acid sequence set forth in amino acids 1 to 8 of SEQ ID NO 7. In an aspect, the composition further comprises one or more pharmaceutically-acceptable excipient. In an aspect, the composition further comprises an agent selected from the group consisting of a diuretic, an angiotensin-converting enzyme (ACE) inhibitor, an angiotensin II receptor blocker (ARB), and a calcium channel blocker (CCB). In an aspect, the composition further comprises furosemide.

The present disclosure also provides nucleic acids encoding selected engineered analogs of ANPs provided herein and compositions thereof. In an aspect, a nucleic acid provided herein encodes a polypeptide comprising the amino acid sequence set forth in any one of SEQ ID NOs: 3-7. In an aspect, a composition provided herein comprises nucleic acid encoding a polypeptide comprising the amino acid sequence set forth in any one of SEQ ID NOs: 3-7. In an aspect, a composition provided herein comprises nucleic acid encoding at least two polypeptides, wherein each of said at least two polypeptides is a polypeptide comprising the amino acid sequence set forth in any one of SEQ ID NOs: 3-7. In an aspect, the composition further comprises an agent selected from the group consisting of a diuretic, an angiotensin-converting enzyme (ACE) inhibitor, an angiotensin II receptor blocker (ARB), and a calcium channel blocker (CCB). In an aspect, the composition further comprises furosemide. In an aspect, the nucleic acid is in the form of a non-viral vector. In an aspect, the non-viral vector is an expression plasmid. In an aspect, the nucleic acid is in the form of a viral vector.

The present disclosure also provides, and includes, methods for treating cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) in a mammal (e.g., a human). For example, compositions that contain one or more selected engineered analogs of ANP provided herein can be administered to a mammal (e.g., human) to reduce blood pressure. For example, compositions that contain one or more selected engineered analog of ANP provided herein can be administered to a mammal (e.g., human) to increase cGMP activation.

The present disclosure provides, and includes, methods of lowering blood pressure, increasing natriuresis, causing arterial vasodilation, suppressing renin and aldosterone, reducing apoptosis, reducing hypertrophy, increasing lusitropy, inducing vascular regeneration, increasing lipolysis, and causing browning of white adipocytes, by administering the compositions provided herein, comprising one or more engineered analogs of ANPs provided herein, that comprise, consist essentially of, or consist of the amino acid sequence set forth in any one of SEQ ID NOs: 3-7.

In an aspect, a method is provided herein for treating a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) in a mammal, wherein said method comprises administering to said mammal a composition comprising a polypeptide comprising the amino acid sequence set forth in any one of SEQ ID NOs: 3-7 or nucleic acid encoding said polypeptide. In an aspect, the mammal is a human. In an aspect, the cardiovascular, cardiorenal, or metabolic disease is hypertension. In an aspect, the cardiovascular, cardiorenal, or metabolic disease is resistant hypertension. In an aspect, the composition comprises an agent selected from the group consisting of a diuretic, an angiotensin-converting enzyme (ACE) inhibitor, an angiotensin II receptor blocker (ARB), and a calcium channel blocker (CCB). In an aspect, the composition comprises furosemide.

In an aspect, a method is provided herein for treating a mammal at risk of developing a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH), wherein said method comprises administering to said mammal a composition comprising a polypeptide comprising the amino acid sequence set forth in any one of SEQ ID NOs: 3-7 or nucleic acid encoding said polypeptide. In an aspect, the mammal is a human. In an aspect, the cardiovascular, cardiorenal, or metabolic disease is hypertension. In an aspect, the cardiovascular, cardiorenal, or metabolic disease is resistant hypertension. In an aspect, the composition comprises an agent selected from the group consisting of a diuretic, an angiotensin-converting enzyme (ACE) inhibitor, an angiotensin II receptor blocker (ARB), and a calcium channel blocker (CCB). In an aspect, the composition comprises furosemide.

In an aspect, a method is provided herein for alleviating the symptoms of a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) in a mammal, wherein said method comprises administering to said mammal a composition comprising a polypeptide comprising the amino acid sequence set forth in any one of SEQ ID NOs: 3-7 or nucleic acid encoding said polypeptide. In an aspect, the mammal is a human. In an aspect, the cardiovascular, cardiorenal, or metabolic disease is hypertension. In an aspect, the cardiovascular, cardiorenal, or metabolic disease is resistant hypertension. In an aspect, the composition comprises an agent selected from the group consisting of a diuretic, an angiotensin-converting enzyme (ACE) inhibitor, an angiotensin II receptor blocker (ARB), and a calcium channel blocker (CCB). In an aspect, the composition comprises furosemide.

### DESCRIPTION OF THE DRAWINGS

Aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIG. 1A illustrates the structure of ANP.

FIG. 1B illustrates the structure of CRRL 191.

FIG. 2 is a plot of cGMP production levels over 6 hours in HEK293/GCA overexpressing cells in response to ANP or CRRL 191 at a dose of 10⁻⁸M.

FIG. 3 is a plot of in vitro degradation of CRRL 191 and ANP in a neprilysin assay.

FIG. 4 is a plot of cGMP production levels in primary human cardiomyocytes induced by ANP and CRRL 191.

FIG. 5 is a plot of cGMP production levels in primary human cardiomyocytes induced by MANP, CRRL 191, and CRRL 101.

FIG. 6 is a plot of apoptosis reductions by increasing doses of CRRL 191 compared to control over 90 hours using real time imaging in primary human cardiomyocytes induced by CRRL 191.

FIG. 7A is a plot of binding kinetics of ANP to GC-A measured by surface plasmon resonance (SPR).

FIG. 7B is a plot of binding kinetics of MANP to GC-A measured by surface plasmon resonance (SPR).

FIG. 7C is a plot of binding kinetics of CRRL 191 to GC-A measured by surface plasmon resonance (SPR).

FIG. 8A is a comparison of systolic blood pressure reduction induced by saline vehicle, 100 pmol/kg/min or 300 pmol/kg/min of CRRL 191, or 100 pmol/kg/min or 300 pmol/kg/min of ANP in spontaneously hypertensive rats during infusion and during washout.

FIG. 8B is a comparison of diastolic blood pressure reduction induced by saline, 100 pmol/kg/min or 300 pmol/kg/min of CRRL 191, or 100 pmol/kg/min or 300 pmol/kg/min of ANP in spontaneously hypertensive rats during infusion and during washout.

FIG. 8C is a comparison of mean arterial blood pressure reduction induced by saline, 100 pmol/kg/min or 300 pmol/kg/min of CRRL 191, or 100 pmol/kg/min or 300 pmol/kg/min of ANP in spontaneously hypertensive rats during infusion and during washout.

FIG. 9A is a comparison of level of plasma cGMP induced by saline, 100 pmol/kg/min or 300 pmol/kg/min of CRRL 191, or 100 pmol/kg/min or 300 pmol/kg/min of ANP in spontaneously hypertensive rats during infusion and during washout.

FIG. 9B is a comparison of change in plasma cGMP level induced by saline, 100 pmol/kg/min or 300 pmol/kg/min of CRRL 191, or 100 pmol/kg/min or 300 pmol/kg/min of ANP in spontaneously hypertensive rats during infusion and during washout.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well known structures, interfaces, and processes have not been shown in detail in order not to unnecessarily obscure the present disclosure. It is intended that no part of this specification be construed to effect a disavowal of any part of the full scope of the present disclosure. Hence, the following descriptions are intended to illustrate some particular embodiments of the disclosure, and not to exhaustively specify all permutations, combinations, and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art. In case of conflict, the present specification, including definitions, will control.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein include and comprise one or more steps or actions for achieving the described method. The method steps and/or actions may be interchanged with one another without departing from the scope of the present disclosure. In other words, unless a specific order of steps or actions is required for proper operation of the embodiment, the order and/or use of specific steps and/or actions may be modified without departing from the scope of the present disclosure. Although methods and materials similar or equivalent to those described herein may also be used to practice the aspects of present disclosure, suitable methods and materials are described herein.

As used in the description of the disclosure and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a duration and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y As used herein, phrases such as “between about X and Y” mean “between about X and about Y” and phrases such as “from about X to Y” mean “from about X to about Y.”

As used herein, the term “exemplary” is used to mean serving as an example, instance, or illustration. Any aspect or aspect described as “exemplary” is not necessarily to be construed as preferred or advantageous over other aspects or aspects, nor is it meant to preclude equivalent structures and techniques known to those of ordinary skill in the art. Rather, use of the word exemplary is intended to present concepts in a concrete fashion, and the disclosed subject matter is not limited by such examples.

### Polypeptides

This disclosure provides methods and materials related to selected engineered analogs of atrial natriuretic peptides (ANPs). For example, this disclosure provides the polypeptides set forth in Table 1. In an aspect, a selected engineered analog of ANP provided herein is a polypeptide that comprises the amino acid sequence set forth in any one of SEQ ID NOs: 3-7. In an aspect, a selected engineered analog of ANP provided herein is a polypeptide that consists essentially of the amino acid sequence set forth in any one of SEQ ID NOs: 3-7. In an aspect, a selected engineered analog of ANP provided herein is a polypeptide that consists of the amino acid sequence set forth in any one of SEQ ID NOs: 3-7. In an aspect, a selected engineered analog of ANP comprises the amino acid sequence set for in SEQ ID NO: 3. In an aspect, a selected engineered analog of ANP consists essentially of the amino acid sequence set for in SEQ ID NO: 3. In an aspect, a selected engineered analog of ANP consists of the amino acid sequence set for in SEQ ID NO: 3. In an aspect, a selected engineered analog of ANP is CRRL 101. In an aspect, a selected engineered analog of ANP comprises the amino acid sequence set for in SEQ ID NO: 4. In an aspect, a selected engineered analog of ANP consists essentially of the amino acid sequence set for in SEQ ID NO: 4. In an aspect, a selected engineered analog of ANP consists of the amino acid sequence set for in SEQ ID NO: 4. In an aspect, a selected engineered analog of ANP is CRRL 90. In an aspect, a selected engineered analog of ANP comprises the amino acid sequence set for in SEQ ID NO: 5. In an aspect, a selected engineered analog of ANP consists essentially of the amino acid sequence set for in SEQ ID NO: 5. In an aspect, a selected engineered analog of ANP consists of the amino acid sequence set for in SEQ ID NO: 5. In an aspect, a selected engineered analog of ANP is CRRL 91. In an aspect, a selected engineered analog of ANP comprises the amino acid sequence set for in SEQ ID NO: 6. In an aspect, a selected engineered analog of ANP consists essentially of the amino acid sequence set for in SEQ ID NO: 6. In an aspect, a selected engineered analog of ANP consists of the amino acid sequence set for in SEQ ID NO: 6. In an aspect, a selected engineered analog of ANP is CRRL 111. In an aspect, a selected engineered analog of ANP comprises, consists essentially of, or consists of the amino acid sequence set for in SEQ ID NO: 7. In an aspect, a selected engineered analog of ANP comprises the amino acid sequence set for in SEQ ID NO: 7. In an aspect, a selected engineered analog of ANP consists essentially of the amino acid sequence set for in SEQ ID NO: 7. In an aspect, a selected engineered analog of ANP consists of the amino acid sequence set for in SEQ ID NO: 7. In an aspect, a selected engineered analog of ANP is CRRL 191.

TABLE 1
Exemplary engineered modified ANP polypeptide

| SEQ | | |
|---|---|---|
| ID NO | Sequence | Name |
| 1 | SLRRSSCFGGRMDRIGAQSGLGCNSFRY | ANP |
| 2 | SLRRSSCFGGRMDRIGAQSGLGCNSFRYRITAREDKQGWA | MANP |
| 3 | SLRRSSCFGHKIDRINHVSNLGCNSFRYRITAREDKQGWA | CRRL 101 |
| 4 | SLRRSSCFGHKIDRINHVSSNLGCNSFRYRITAREDKQGWA | CRRL 90 |
| 5 | SLRRSSCFGHKIDRINHVSNNLGCNSFRYRITAREDKQGWA | CRRL 91 |
| 6 | TAPRSLRRSSCFGHKIDRINHVSNLGCNSFRYRITAREDKQGWA | CRRL 111 |
| 7 | RALLTAPRSLRRSSCFGHKIDRINHVSNLGCNSFRYRITAREDKQGWA | CRRL 191 |

Without being bound by theory, selected engineered analogs of ANP with amino acid substitutions, additions, and deletions, may function similarly to each other. In an aspect, a selected engineered analog of ANP provided herein may comprise, consist essentially of, or consist of the amino acid sequence set forth in any one of SEQ ID NOs: 1-7 having zero, one, two, or three amino acid substitutions within the articulated sequence of the sequence identifier (e.g., SEQ ID NO:1).

In an aspect, a selected engineered analog of ANP provided herein can include one, two, or three conservative substitutions. Without being bound by theory, conservative amino acid substitutions can be made by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Non-limiting examples of useful conservative substitutions can include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. In an aspect, a selected engineered analog of ANP provided herein can include one, two, or three conservative substitutions, wherein an acidic amino acid residue is substituted by another acidic amino acid residue. In an aspect, a selected engineered analog of ANP provided herein can include one, two, or three conservative substitutions, wherein a basic amino acid residue is substituted by another basic amino acid residue.

In an aspect, a selected engineered analog of ANP provided herein can include one, two, or three non-conservative substitutions. Without being bound by theory, non-conservative substitutions typically entail exchanging a member of one of the classes described above for a member of another class. Such production can be desirable to provide large quantities or alternative embodiments of such compounds. Whether an amino acid change results in a functional polypeptide can readily be determined by assaying the specific activity of the peptide variant using, for example, methods disclosed herein.

In an aspect, a selected engineered analog of ANP provided herein that comprises, consists essentially of, or consists of the amino acid sequence set forth in any one of SEQ ID NOs: 1-7 is a polypeptide that has zero, one, two, or three amino acid additions within the articulated sequence of the sequence identifier (e.g., SEQ ID NO:1). In an aspect, a selected engineered analog of ANP provided herein that comprises, consists essentially of, or consists of the amino acid sequence set forth in any one of SEQ ID NOs:1-7 is a polypeptide that has zero, one, two, or three amino acid deletions within the articulated sequence of the sequence identifier (e.g., SEQ ID NO:1). In an aspect, a selected engineered analog of ANP provided herein that comprises, consists essentially of, or consists of the amino acid sequence set forth in any one of SEQ ID NOs:1-7 is a polypeptide that has zero, one, two, three, four, or five amino acid residues preceding the articulated sequence of the sequence identifier (e.g., SEQ ID NO:1). In an aspect, a selected engineered analog of ANP provided herein that comprises, consists essentially of, or consists of the amino acid sequence set forth in any one of SEQ ID NOs:1-7 is a polypeptide that has zero, one, two, three, four, or five amino acid residues following the articulated sequence of the sequence identifier (e.g., SEQ ID NO:1).

In an aspect, a selected engineered analog of ANP provided herein that comprises, consists essentially of, or consists of the amino acid sequence set forth in any one of SEQ ID NOs:1-7 is a polypeptide that has zero, one, two, or three amino acid substitutions within the articulated sequence of the sequence identifier (e.g., SEQ ID NO:1), and/or zero, one, two, or three amino acid additions within the articulated sequence of the sequence identifier (e.g., SEQ ID NO:1), and/or zero, one, two, or three amino acid deletions within the articulated sequence of the sequence identifier (e.g., SEQ ID NO:1), and/or zero, one, two, three, four, or five amino acid residues preceding the articulated sequence of the sequence identifier (e.g., SEQ ID NO:1), and/or zero, one, two, three, four, or five amino acid residues following the articulated sequence of the sequence identifier (e.g., SEQ ID NO: 1).

Without being bound by theory, certain amino acids of the selected engineered analogs of ANP provided herein are not modified (e.g., amino acid substitutions, additions, deletions) as provide beneficial properties. For example, the cysteine residues are used to form disulfide bridges. In an aspect, the cysteine residues of a selected engineered analog of ANP provided herein are not substituted. In an aspect, the first four amino acids of SEQ ID NO: 6 (e.g., the amino acids TAPR) are not modified (e.g., amino acid substitutions, additions, deletions). In an aspect, the first four amino acids of SEQ ID NO: 7 (e.g., the amino acids RALL) are not modified (e.g., amino acid substitutions, additions, deletions). In an aspect, the first eight amino acids of SEQ ID NO: 7 (e.g., the amino acids RALLTAPR) are not modified (e.g., amino acid substitutions, additions, deletions). The majority of naturally occurring amino acids are L-amino acids, and naturally occurring polypeptides are largely comprised of L-amino acids. D-amino acids are the enantiomers of L-amino acids. In an aspect, a polypeptide as provided herein can contain one or more D-amino acids.

In an aspect, a selected engineered analog of ANP provided herein has at least 70%, at least 75%, at least 80%, at least 85%, at least 90%, at least 92%, at least 95%, at least 97%, or at least 99% sequence identity to the amino acid sequence set forth in any one of SEQ ID NOs: 1-7. Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number can be determined by methods known in the art, for example, as disclosed in WO/2018/089601, which is incorporated by reference herein in its entirety.

The selected engineered analogs of ANP provided herein can be produced using any suitable methods, including but not limited to, solution phases synthesis (SPS), solid phase peptide synthesis (SPPS), and expressed protein ligation (EPL). The selected engineered analogs of ANP provided herein can be generated using manual techniques or automated techniques (e.g., using an Applied BioSystems (Foster City, CA) Peptide Synthesizer or a Biosearch Inc. (San Rafael, CA) automatic peptide synthesizer). The selected engineered analogs of ANP provided herein can also be produced recombinantly.

The selected engineered analogs of ANP provided herein typically are cyclic due to disulfide bonds between the cysteine residues underlined in the sequences shown in Table 1. Disulfide bonds can be formed by any methods known to the skilled artisan, including but not limited to, oxidation (either enzyme-mediated or via the reactions of low-molecular-mass oxidants) or reagents (e.g., dicarbonyl and related crosslinking agents, such as glutaraldehyde). Disulfide bonds between cysteine residues can also be introduced by mild oxidation of the linear polypeptides using KCN as taught, e.g., in U.S. Pat. No. 4,757,048.

The term “substantially pure” as used herein with reference to a polypeptide means the polypeptide is substantially free of other polypeptides, lipids, carbohydrates, and nucleic acid with which it is naturally associated. Thus, a substantially pure polypeptide is any polypeptide that is removed from its natural environment and is at least 60 percent pure. A substantially pure polypeptide can be at least about 65, 70, 75, 80, 85, 90, 95, or 99 percent pure. Typically, a substantially pure polypeptide will yield a single major band on a non-reducing polyacrylamide gel. In an aspect, a substantially pure polypeptide provided herein is a polypeptide that is synthesized to have a purity of at least about 60, 65, 70, 75, 80, 85, 90, 95, or 99 percent.

Any method can be used to obtain a substantially pure polypeptide. For example, polypeptide purification techniques, such as affinity chromatography and HPLC, as well as polypeptide synthesis techniques can be used. In addition, any material can be used as a source to obtain a substantially pure polypeptide. For example, tissue from wild-type or transgenic animals can be used as a source material. In addition, tissue culture cells engineered to over-express a particular polypeptide can be used to obtain substantially pure polypeptide. Solid phase peptide synthesis can also be used to a obtain substantially pure polypeptide. Further, a polypeptide can be engineered to contain an amino acid sequence that allows the polypeptide to be captured onto an affinity matrix. For example, a tag such as c-myc, hemagglutinin, polyhistidine, or FLAG™ tag (Kodak) can be used to aid polypeptide purification. Such tags can be inserted anywhere within the polypeptide including at either the carboxyl or amino termini, or in between. Other fusions that can be used include enzymes that aid in the detection of the polypeptide, such as alkaline phosphatase.

Without being bound by theory, a selected engineered analog of ANP is processed from pro-peptides. A selected engineered analog of ANP provided herein can be processed, in part, from any pro-peptide. In an aspect, a selected engineered analog of ANP can be manufactured as a pro-peptide that is further processed, naturally or non-naturally, into a selected engineered analog of ANP provided herein.

Without being bound by theory, the engineered analogs of ANP provided herein can function through one or more of the guanylyl cyclase receptors through which the native natriuretic polypeptides function. For example, the polypeptides provided herein can bind to and function through the GC-A (also known as NPR-A) receptor through which ANP, BNP, and DNP function, although they also may function through the GC-B (also known as NPR-B) receptor through which CNP functions. In an aspect, an engineered analogs of ANP provided herein can bind to guanyl cyclase-A (GC-A) receptor. In an aspect, an engineered analog of ANP provided herein can bind to guanyl cyclase-A (GC-A) receptor. In an aspect, an engineered analog of ANP provided herein can bind to and function through more than one guanylyl cyclase receptor, including GC-A and GB-B. Methods for evaluating which receptor is involved in function of a particular engineered analog of ANP are known in the art. For example, glomeruli, which contain both GC-A and GC-B, can be isolated (e.g., from a laboratory animal such as a dog) and incubated with an engineered analog of ANP (e.g., any one of SEQ ID NO: 3-7), and cGMP levels can be measured. Glomeruli can be pretreated with antagonists of GC-A or GC-B to determine whether cGMP production stimulated by a natriuretic polypeptide through one or the other receptor can be attenuated.

The biological activity of the selected engineered analogs of ANP provided herein can be determined using any of a number of assays, including those described herein. For example, the activity of an engineered analog of ANP provided can be determined in vitro by testing its effect on cGMP production in cultured cells (e.g., cultured cardiac fibroblasts, aortic endothelial cells, or glomerular cells). Cells can be exposed to an engineered analog of ANP provided herein (e.g., 10⁻¹⁰ to 10⁻⁴ M of CRRL 191), and samples can be assayed to evaluate the effects on cGMP generation. cGMP generation can be detected and measured using, for example, a competitive RIA cGMP kit (Perkin-Elmer, Boston, MA) or a cGMP ELISA kit (Enzo Life Sciences, Farmingdale, NY).

A selected engineered analog of ANP provided herein can be any appropriate length (e.g., can include any number of amino acids). For example, a selected engineered analog of ANP provided herein can be from about 20 amino acids in length to about 100 amino acids (e.g., from about 20 to about 90 amino acids, from about 20 to about 80 amino acids, from about 20 to about 70 amino acids, from about 20 to about 60 amino acids, from about 20 to about 55 amino acids, from about 20 to about 52 amino acids, from about 20 to about 50 amino acids, from about 20 to about 100 amino acids, from about 30 to about 100 amino acids, from about 40 to about 100 amino acids, from about 50 to about 100 amino acids, from about 60 to about 100 amino acids, from about 70 to about 100 amino acids, from about 80 to about 100 amino acids, from about 90 to about 100 amino acids, from about 20 to about 80 amino acids, from about 20 to about 70 amino acids, from about 30 to about 60 amino acids, from about 35 to about 55 amino acids, from about 38 to about 52 amino acids, from about 40 to about 50 amino acids, or from about 45 to about 50 amino acids) in length. In an aspect, a selected engineered analog of ANP provided herein can be from about 46 to about 50 amino acid sequences in length. For example, a selected engineered analog of ANP provided herein and having GC-A binding properties can be from about 38 to about 42 amino acid sequences in length. For example, a selected engineered analog of ANP provided herein and having GC-A binding properties can be from about 42 to about 46 amino acid sequences in length. For example, a selected engineered analog of ANP provided herein and having GC-A binding properties can be from about 46 to about 50 amino acid sequences in length.

A selected engineered analog of ANP provided herein can comprise one or more sequences present in a polypeptide having natriuretic polypeptide activity (e.g., ANP, BNP, CNP, urodilatin, and DNP). In an aspect, a selected engineered analog of ANP can comprise a non-naturally occurring sequence. In an aspect, a selected engineered analog of ANP can comprise a naturally occurring sequence and a non-naturally occurring sequence. In an aspect, a selected engineered analog of ANP can comprise a sequence present in any species, including, but not limited to, human, horse, pig, goat, cow, dog, cat, rat, or snake.

### Nucleic Acids

The present disclosure also provides nucleic acid encoding one or more selected engineered analogs of ANP provided herein (e.g., one or more polypeptides that comprises, consists essentially of, or consists of the amino acid sequence set forth in any one of SEQ ID NOs: 3-7). For example, the present disclosure provides vectors (e.g., plasmids and viral vectors) that include nucleic acid encoding one or more engineered analogs of ANP provided herein (e.g., one or more polypeptides that comprises, consists essentially of, or consists of the amino acid sequence set forth in any one of SEQ ID NOs: 3-7) in a manner such that the polypeptide can be expressed within a cell.

When a vector including nucleic acid encoding one or more selected engineered analogs of ANP provided herein (e.g., one or more polypeptides that comprises, consists essentially of, or consists of the amino acid sequence set forth in any one of SEQ ID NOs: 3-7) is a non-viral vector, any appropriate non-viral vector can be used. In an aspect, a non-viral vector can be an expression plasmid (e.g., a cDNA expression vector).

When a vector including nucleic acid encoding one or more S selected engineered analogs of ANP provided herein (e.g., one or more polypeptides that comprises, consists essentially of, or consists of the amino acid sequence set forth in any one of SEQ ID NOs: 3-7) is a viral vector, any appropriate viral vector can be used. In an aspect, a viral vector can be derived from a positive-strand virus or a negative-strand virus. In an aspect, a viral vector can be derived from a virus having a single-stranded genome or a virus having a double stranded genome. In an aspect, a viral vector can be derived from a virus with a DNA genome or a RNA genome. In an aspect, a viral vector can be a chimeric viral vector. In an aspect, a viral vector can infect dividing cells. In an aspect, a viral vector can infect non-dividing cells. Examples of virus-based vectors that can including nucleic acid encoding a selected engineered analog of ANP provided herein (e.g., a polypeptide that comprises, consists essentially of, or consists of the amino acid sequence set forth in any one of SEQ ID NOs: 3-7) include, without limitation, virus-based vectors based on adenoviruses, adeno-associated viruses (AAVs), retroviruses, lentiviruses, measles viruses, vesicular stomatitis viruses, and vaccinia viruses.

In addition to nucleic acid encoding one or more selected engineered analogs of ANP provided herein (e.g., one or more polypeptides that comprises, consists essentially of, or consists of the amino acid sequence set forth in any one of SEQ ID NOs: 3-7), a vector (e.g., a plasmid or a viral vector) can contain one or more regulatory elements operably linked to the nucleic acid encoding one or more selected engineered analogs of ANP provided herein. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding one or more selected engineered analogs of ANP provided herein (e.g., a polypeptide that comprises, consists essentially of, or consists of the amino acid sequence set forth in any one of SEQ ID NOs: 3-7). In an aspect, a promoter can be a naturally occurring promoter or a recombinant promoter. In an aspect, a promoter can be constitutive or inducible (e.g., in the presence of tetracycline), and can affect the expression of a nucleic acid encoding a polypeptide in a general or cell/tissue-specific manner. Examples of promoters that can be used to drive expression of one or more selected engineered analogs of ANP provided herein (e.g., a polypeptide that comprises, consists essentially of, or consists of the amino acid sequence set forth in any one of SEQ ID NOs: 3-7) in cells include, without limitation, CMV promoters, EF1a promoters, SV40 promoters, PGK1 promoters, Ubc promoters, TRE promoters, and CAG promoters. As used herein, “operably linked” refers to positioning of a regulatory element in a vector relative to a nucleic acid encoding a polypeptide in such a way as to permit or facilitate expression of the encoded polypeptide. For example, a vector can contain a promoter and nucleic acid encoding one or more selected engineered analogs of ANP provided herein. In this case, the promoter is operably linked to a nucleic acid encoding one or more selected engineered analogs of ANP provided herein such that it drives expression of the selected engineered analog(s) of ANP in cells.

### Polypeptide and Nucleic Acid Combinations, Compositions, and Formulations Thereof

This disclosure also provides compositions that include one or more (e.g., one, two, three, four, five, six, or seven) of the selected engineered analogs of ANP provided herein and/or nucleic acid encoding one or more (e.g., one, two, three, four, five, six, or seven) of the selected engineered analogs of ANP provided herein. In an aspect, a composition provided herein comprises, consists essentially of, or consist of one or more of the selected engineered analogs of ANP set forth in Table 1. In an aspect, a composition provided herein comprises one or more nucleic acids encoding the polypeptides that comprise, consist essentially of, or consist of the amino acid sequence set forth in any one of SEQ ID NOs: 3-7. In an aspect, a composition provided herein comprises one or more of the selected engineered analogs of ANP set forth in Table 1 and one or more nucleic acids encoding the polypeptides that comprise, consist essentially of, or consist of the amino acid sequence set forth in any one of SEQ ID NOs: 3-7.

In an aspect, a composition provided herein comprises at least two (e.g., two, three, four, five, six, or seven) of the selected engineered analogs of ANP provided herein and/or nucleic acid encoding at least two (e.g., two, three, four, five, six, seven, eight, nine, ten, 11, 12, 13, 14, 15, 16, 17, 18, 19, 20, or more) of the selected engineered analogs of ANP provided herein. In an aspect, a composition provided herein, comprises, consists essentially of, or consist of at least two of the selected engineered analogs of ANP set forth in Table 1. In an aspect, a composition provided herein comprises at least two of the nucleic acids encoding the polypeptides that comprise, consist essentially of, or consist of the amino acid sequence set forth in any one of SEQ ID NOs: 3-7. In an aspect, a composition provided herein comprises at least two of the selected engineered analogs of ANP set forth in Table 1 and at least two of the nucleic acids encoding the polypeptides that comprise, consist essentially of, or consist of the amino acid sequence set forth in any one of SEQ ID NOs: 3-7.

Any appropriate method can be used to formulate a composition provided herein (e.g., a composition that includes one or more of the selected engineered analogs of ANP provided herein and/or nucleic acid encoding one or more of the selected engineered analogs of ANP provided). In an aspect, the one or more selected engineered analogs of ANP provided herein can be combined with a pharmaceutically acceptable carrier and/or a pharmaceutical excipient. In an aspect, the one or more nucleic acid encoding the polypeptides that comprise, consist essentially of, or consist of the amino acid sequence set forth in any one of SEQ ID NOs: 3-7, can be combined with a pharmaceutically acceptable carrier and/or a pharmaceutical excipient. In an aspect, the one or more selected engineered analogs of ANP provided herein and one or more nucleic acids encoding the polypeptides that comprise, consist essentially of, or consist of the amino acid sequence set forth in any one of SEQ ID NOs: 3-7, can be combined with a pharmaceutically acceptable carrier and/or a pharmaceutical excipient. The term “pharmaceutically acceptable” refers to generally non-toxic, inert, and/or physiologically compatible compounds. A term “pharmaceutical excipient” includes materials such as carriers, pH-adjusting and buffering agents, tonicity adjusting agents, wetting agents, colorants, and preservatives.

In an aspect, the composition provided herein (e.g., a composition that includes one or more of the selected engineered analogs of ANP provided herein and/or nucleic acids encoding one or more of the selected engineered analogs of ANP provided herein) can be formulated with at least one other combination agent chosen from a diuretic, an angiotensin-converting enzyme (ACE) inhibitor, an angiotensin II receptor blocker (ARB), and a calcium channel blocker (CCB). In an aspect, the composition comprises a selected engineered analog of ANP and furosemide.

### Methods of Treatment and Prevention

Without being bound by theory, selected engineered analogs of ANP provided herein can be used to treat, prevent, and/or ameliorate the symptoms of cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH). In an aspect, selected engineered analogs of ANP herein can be used to treat cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH). In an aspect, selected engineered analogs of ANP provided herein can be used to prevent cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH). In an aspect, selected engineered analogs of ANP provided herein can be used to ameliorate the symptoms of cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH). In an aspect, selected engineered analogs of ANP provided herein can be used to treat, prevent, and/or ameliorate the symptoms of hypertension (e.g., resistant hypertension) or heart failure (HF). The presence or extent of disease can be evaluated using methods known in the art, including, without limitation, general clinical examination to evaluate blood pressure, heart rate, heart rhythm, arterial oxygen, and hemoglobin levels; echocardiography to measure ejection fraction, LV and left atrium (LA) diameter, LV wall motion, LV filling pressure, and diastolic function by pulse and tissue Doppler; use of a Swan-Ganz catheter to measure cardiac output, pulmonary wedge capillary pressure, pulmonary arterial pressure, right ventricle pressure, right atrium pressure, and systemic and pulmonary vascular resistance; assessment of kidney function by determination of glomerular filtration rate, serum creatinine, and blood urea nitrogen; and measurement of biomarkers such as BNP, amino-terminal proBNP (NT-proBNP), troponin-T, troponin-I, C-reactive protein (CRP), and creatine-kinase, serum cystatin-C, albuminuria, neutrophil gelatinize associated lopocalin (NGAL), N-acetyl-beta-D-glucosaminidase (NAG), kidney injury molecule-1 (KIM-1), angiotensin-II, renin, aldosterone, and inflammatory cytokines (e.g., interleukin (IL)-6, IL-18, etc.). In an aspect, a selected engineered analog of ANP as provided herein can reduce one or more symptoms of acute or chronic HF, including edema, shortness of breath, and fatigue. To determine the level of efficacy improving symptoms of HF by a selected engineered analog of ANP provided herein, one or more of these parameters can be evaluated (e.g., before and after treatment with the engineered analog of ANP), using methods known in the art, for example. Favorable clinical responses include cardiac unloading (i.e., reduced pressure in the heart), increased glomerular filtration rate (GFR), decreased PRA, decreased levels of angiotensin II, decreased proliferation of cardiac fibroblasts, decreased left ventricular (LV) hypertrophy, decreased LV mass (indicative of reduced fibrosis and hypertrophy), decreased PWCP (an indirect measure of left atrial pressure), decreased right atrial pressure, decreased mean arterial pressure, decreased levels of aldosterone (indicative of an anti-fibrotic effect), decreased ventricular fibrosis, increased ejection fraction, and decreased LV end systolic diameter.

Without being bound by theory, selected engineered analog of ANP provided herein can lower blood pressure, increase natriuresis, cause arterial vasodilation, suppress renin and aldosterone, reduce apoptosis, reduce hypertrophy, increase lusitropy, induce vascular regeneration, increase lipolysis, and cause browning of white adipocytes. In an aspect, the selected engineered analog of ANP can reduce blood pressure in a mammal. In an aspect, selected engineered analogs of ANP provided herein can increase natriuresis in a mammal. In an aspect, selected engineered analogs of ANP provided herein can reduce apoptosis in a mammal. In an aspect, selected engineered analogs of ANP provided herein can cause arterial vasodilation in a mammal. In an aspect, selected engineered analogs of ANP provided herein can suppress renin and aldosterone in a mammal. In an aspect, selected engineered analogs of ANP provided herein can increase lusitropy in a mammal. In an aspect, selected engineered analogs of ANP provided herein can induce vascular regeneration in a mammal. In an aspect, selected engineered analogs of ANP provided herein can increase lipolysis in a mammal. In an aspect, selected engineered analogs of ANP provided herein can cause browning of white adipocytes in a mammal.

The activity of a selected engineered analog of ANP also can be evaluated in vivo by, for example, testing its effects on factors such as plasma cGMP levels, urinary cGMP excretion, net renal generation of cGMP, glomerular filtration rate, blood pressure, heart rate, hemodynamic function such as cardiac output, pulmonary wedge pressure, systemic vascular resistance, and renal function such as renal blood flow, urine volume, and sodium excretion rate in a mammal (e.g., a rodent, pig, sheep, dog, or human). In an aspect, such parameters can be evaluated after inducing heart failure (e.g., by rapid right ventricular pacing) or hypertension.

In an aspect, one or more selected engineered analogs of ANP provided herein (e.g., polypeptides that comprise, consist essentially of, or consist of the amino acid sequence set forth in any of SEQ ID NOs: 3-7) and/or nucleic acid encoding one or more of the selected engineered analogs of ANP provided herein provided herein (e.g., nucleic acid encoding one or more selected engineered analogs of ANP provided herein that comprise, consist essentially of, or consist of the amino acid sequence set forth in any of SEQ ID NOs: 3-7) can be administered to a mammal (e.g., human) to treat, prevent, and/or alleviate the symptoms of cardiovascular disease (e.g., hypertension, heart failure, cardiomyopathies, valvular disease, myocardial infarction) within the mammal. In an aspect, one or more selected engineered analogs of ANP provided herein (e.g., polypeptides that comprise, consist essentially of, or consist of the amino acid sequence set forth in any of SEQ ID NOs: 3-7) and/or nucleic acid encoding one or more of the selected engineered analogs of ANP provided herein provided herein (e.g., nucleic acid encoding one or more selected engineered analogs of ANP provided herein that comprise, consist essentially of, or consist of the amino acid sequence set forth in any of SEQ ID NOs: 3-7) can be administered to a mammal (e.g., human) to treat, prevent, and/or alleviate the symptoms of cardiorenal disease (e.g., acute kidney injury, chronic kidney disease, polycystic kidney disease, chemotherapeutic-induced cardiac and renal dysfunction) within the mammal. In an aspect, one or more selected engineered analogs of ANP provided herein (e.g., polypeptides that comprise, consist essentially of, or consist of the amino acid sequence set forth in any of SEQ ID NOs: 3-7) and/or nucleic acid encoding one or more of the selected engineered analogs of ANP provided herein provided herein (e.g., nucleic acid encoding one or more selected engineered analogs of ANP provided herein that comprise, consist essentially of, or consist of the amino acid sequence set forth in any of SEQ ID NOs: 3-7) can be administered to a mammal (e.g., human) to treat, prevent, and/or alleviate the symptoms of metabolic disease (e.g., diabetes, obesity, metabolic syndrome, hyperlipidemia, insulin resistance, primary aldosteronism, NASH) within the mammal.

As used herein, the terms “treat” or “treatment” is an approach for obtaining beneficial or desired clinical results. In an aspect, the terms “treat” or “treatment” means to administer one or more selected engineered analogs of ANP disclosed herein that partially or completely alleviates, ameliorates, relieves, inhibits, delays onset of, reduces severity of, and/or reduces incidence of one or more symptoms, features and causes of a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH). The terms “treat” or “treatment” includes the administration of one or more selected engineered analogs of ANP disclosed herein to prevent or delay the onset of a symptom, complication, or biochemical indicia of a cardiovascular, cardiorenal, or metabolic disease, alleviating a symptom or arresting or inhibiting further development of a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH). Treatment may be prophylactic (to prevent or delay the onset of the cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH), or to prevent the manifestation of a clinical or subclinical symptom thereof) or therapeutic suppression or alleviation of a symptom after the manifestation of the cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH).

Any appropriate mammal can be administered one or more selected engineered analogs of ANP provided herein (e.g., one or more selected engineered analogs of ANP that comprise, consist essentially of, or consist of the amino acid sequence set forth in any of SEQ ID NOs: 3-7) and/or nucleic acid encoding one or more selected engineered analogs of ANP provided herein (e.g., nucleic acid encoding one or more selected engineered analogs of ANP that comprise, consist essentially of, or consist of the amino acid sequence set forth in any of SEQ ID NOs: 3-7) to treat that mammal. Examples of mammals that can be administered one or more selected engineered analogs of ANP provided herein (and/or nucleic acid encoding one or more selected engineered analogs of ANP provided herein) include, without limitation, humans, non-human primates (e.g., monkeys or apes), horses, dogs, cats, bovine species, pigs, sheep, mice, rats, hamsters, bats, foxes, goats, mink, and deer. In an aspect, a human identified as having or as being at risk of developing a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) can be administered one or more selected engineered analogs of ANP provided herein to treat that human. In an aspect, a human identified as having or as being at risk of developing a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) can be administered nucleic acids encoding one or more selected engineered analogs of ANP provided herein to treat that human. In an aspect, a human identified as having or as being at risk of developing a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) can be administered one or more selected engineered analogs of ANP provided herein and nucleic acids encoding one or more selected engineered analogs of ANP provided herein to treat that human.

In an aspect, the efficacy of the treatment is measured by clinical parameters such as a reduction of symptoms. Symptoms of cardiovascular, cardiorenal, or metabolic disease include, but are not limited to, edema, shortness of breath, and fatigue, as well as cardiac unloading (i.e., reduced pressure in the heart), increased glomerular filtration rate (GFR), decreased PRA, decreased levels of angiotensin II, decreased proliferation of cardiac fibroblasts, decreased left ventricular (LV) hypertrophy, decreased LV mass (indicative of reduced fibrosis and hypertrophy), decreased PWCP (an indirect measure of left atrial pressure), decreased right atrial pressure, decreased mean arterial pressure, decreased levels of aldosterone (indicative of an anti-fibrotic effect), decreased ventricular fibrosis, increased ejection fraction, and decreased LV end systolic diameter.

In an aspect, the materials and methods described herein can be used to delay the onset of one or more symptoms of cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) within a mammal at risk of developing cardiovascular, cardiorenal, or metabolic disease cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) by, for example, 10, 20, 30, 40, 50, 60, 70, 80, 90, 95, or more percent. In an aspect, the methods and materials described herein can be used to reduce the duration and/or the severity of one or more symptoms of one or more symptoms of cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) present within a mammal having a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) by, for example, 10, 20, 30, 40, 50, 60, 70, 80, 90, 95, or more percent. See, e.g., J Jamison et al., Harrison's Principles of Medicine (20^{th} Edition 2018).

When administering a composition provided herein to a mammal (e.g., a human), any appropriate route of administration can be used. In an aspect, a composition provided herein can be administered to a mammal (e.g., a human) intramuscularly (e.g., via intramuscular injection), subcutaneously (e.g., via a subcutaneous injection), orally, intranasally, transcutaneously, or via inhalation. In an aspect, the route and/or mode of administration of a composition provided herein can be adjusted for the mammal being treated. In an aspect, different doses of a composition provided herein are administered to a mammal by the same route of administration. In an aspect, different doses of a composition provided herein are administered to a mammal by different routes of administration. For example, a first dose of one or more selected engineered analogs of ANP provided herein (e.g., one or more selected engineered analogs of ANP that comprise, consist essentially of, or consist of the amino acid sequence set forth in any of SEQ ID NOs: 3-7) may be administered subcutaneously while the second dose is administered intranasally.

In an aspect, an effective amount of one or more selected engineered analogs of ANP provided herein (e.g., one or more selected engineered analogs of ANP that comprise, consist essentially of, or consist of the amino acid sequence set forth in any of SEQ ID NOs: 3-7) can be an amount that reduces a symptom (e.g., shortness of breath, systolic pressure, diastolic pressure) within the mammal (e.g., a human) without producing significant toxicity to the mammal. In an aspect, an effective amount of nucleic acids encoding one or more selected engineered analogs of ANP provided herein (e.g., nucleic acid encoding one or more selected engineered analogs of ANP that comprise, consist essentially of, or consist of the amino acid sequence set forth in any of SEQ ID NOs: 3-7) can be an amount that reduces a symptom (e.g., shortness of breath, systolic pressure, diastolic pressure) within the mammal (e.g., a human) without producing significant toxicity to the mammal. In an aspect, an effective amount of one or more selected engineered analogs of ANP provided herein (e.g., one or more selected engineered analogs of ANP that comprise, consist essentially of, or consist of the amino acid sequence set forth in any of SEQ ID NOs: 3-7) and nucleic acids encoding one or more selected engineered analogs of ANP provided herein (e.g., nucleic acid encoding one or more selected engineered analogs of ANP that comprise, consist essentially of, or consist of the amino acid sequence set forth in any of SEQ ID NOs: 3-7) can be an amount that reduces a symptom (e.g., shortness of breath, systolic pressure, diastolic pressure) within the mammal (e.g., a human) without producing significant toxicity to the mammal. The effective amount can remain constant or can be adjusted as a sliding scale or variable dose depending on the mammal's response to treatment. Without being bound by theory, various factors can influence the actual effective amount used for a particular application. The dose may be adjusted by the skilled artisan or treating physician.

In an aspect, an effective amount of one or more selected engineered analogs of ANP provided herein (e.g., one or more selected engineered analogs of ANP that comprise, consist essentially of, or consist of the amino acid sequence set forth in any of SEQ ID NOs: 3-7) is about 1.0 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90, or 100 mg. In an aspect, an effective amount of one or more selected engineered analogs of ANP provided herein (e.g., one or more selected engineered analogs of ANP that comprise, consist essentially of, or consist of the amino acid sequence set forth in any of SEQ ID NOs: 3-7) is about 1 mg-100 mg, about 1 mg-80 mg, about 0.5 mg-80 mg, about 0.5 mg-60 mg, or about 1 mg-50 mg. In an aspect, an oral dose of one or more selected engineered analogs of ANP provided herein (e.g., one or more selected engineered analogs of ANP that comprise, consist essentially of, or consist of the amino acid sequence set forth in any of SEQ ID NOs: 3-7) is about 1 mg-100 mg, about 1 mg-80 mg, about 0.5 mg-80 mg, about 0.5 mg-60 mg, or about 1 mg-50 mg. In an aspect, an effective amount given by oral administration of one or more selected engineered analogs of ANP provided herein (e.g., one or more selected engineered analogs of ANP that comprise, consist essentially of, or consist of the amino acid sequence set forth in any of SEQ ID NOs: 3-7) is 1 mg-50 mg. In an aspect, an effective amount given by subcutaneous administration of one or more selected engineered analogs of ANP provided herein (e.g., one or more selected engineered analogs of ANP that comprise, consist essentially of, or consist of the amino acid sequence set forth in any of SEQ ID NOs: 3-7) is about 0.01 μg/kg-50 μg/kg, about 0.1 μg/kg-40 μg/kg, about 0.1 μg/kg-30 μg/kg, about 0.05 μg/kg-40 μg/kg, or about 0.05 μg/kg-30 μg/kg.

In an aspect, an effective frequency of administration of one or more selected engineered analogs of ANP provided herein (e.g., one or more selected engineered analogs of ANP that comprise, consist essentially of, or consist of the amino acid sequence set forth in any of SEQ ID NOs: 3-7) and/or nucleic acid encoding one or more selected engineered analogs of ANP provided herein (e.g., nucleic acid encoding one or more selected engineered analogs of ANP that comprise, consist essentially of, or consist of the amino acid sequence set forth in any of SEQ ID NOs: 3-7) can be a frequency that reduces a symptom (e.g., shortness of breath, systolic pressure, diastolic pressure) within the mammal (e.g., a human) without producing significant toxicity to the mammal. In an aspect, an effective frequency of administration of one or more selected engineered analogs of ANP provided herein (and/or nucleic acid encoding one or more selected engineered analogs of ANP provided herein) can be from about once every two hours, about once every three hours, about once every four hours, about once every five hours, about once every six hours, about once every seven hours, about once every eight hours, about once every nine hours, about once every ten hours, about once every eleven hours, about once every twelve hours, about once every day, about once every two days, about once every three days, about once every four days, about once every five days, about once every six days, about once every week, about once every two weeks, about once every three weeks, about once every four weeks, about once a month, about once every two months, about once every three months, about once every four months, about once every five months, about once every six months, or about once a year. Without being bound by theory, various factors can influence the actual effective frequency used for a particular application. The frequency may be adjusted by the skilled artisan or treating physician.

In an aspect, an effective duration of administration of one or more selected engineered analogs of ANP provided herein (e.g., one or more selected engineered analogs of ANP that comprise, consist essentially of, or consist of the amino acid sequence set forth in any of SEQ ID NOs: 3-7) and/or nucleic acid encoding one or more selected engineered analogs of ANP provided herein (e.g., nucleic acid encoding one or more selected engineered analogs of ANP that comprise, consist essentially of, or consist of the amino acid sequence set forth in any of SEQ ID NOs: 3-7) can be a duration that reduces a symptom (e.g., shortness of breath, systolic pressure, diastolic pressure) within the mammal (e.g., a human) without producing significant toxicity to the mammal. In an aspect, an effective frequency of administration of one or more selected engineered analogs of ANP provided herein (and/or nucleic acid encoding one or more selected engineered analogs of ANP provided herein) can be from about one day, two days, three days, four days, five days, six days, seven days, two weeks, three weeks, four weeks, one month, two months, three months, four months, five months, six months, one year, two years, three years, four years, five years, ten years, or as long as needed. Without being bound by theory, various factors can influence the actual effective duration used for a particular application. The duration may be adjusted by the skilled artisan or treating physician.

The present disclosure also provides kits comprising one or more (e.g., one, two, three, four, five, six, seven, eight, nine, ten, 11, 12, 13, 14, 15, 16, 17, 18, 19, 20, or more) selected engineered analogs of ANP provided herein (e.g., one or more substantially pure selected engineered analogs of ANP that comprise, consist essentially of, or consist of the amino acid sequence set forth in any of SEQ ID NOs: 3-7) and/or nucleic acid encoding one or more selected engineered analogs of ANP provided herein (e.g., nucleic acid encoding one or more selected engineered analogs of ANP that comprise, consist essentially of, or consist of the amino acid sequence set forth in any of SEQ ID NOs: 3-7). In an aspect, a kit provided herein comprises one or more of the selected engineered analogs of ANP provided herein (e.g., one or more substantially pure selected engineered analogs of ANP that comprise, consist essentially of, or consist of the amino acid sequence set forth in any of SEQ ID NOs: 3-7). In an aspect, a kit provided herein comprises nucleic acids encoding one or more selected engineered analogs of ANP provided herein (e.g., nucleic acids encoding one or more selected engineered analogs of ANP that comprise, consist essentially of, or consist of the amino acid sequence set forth in any of SEQ ID NOs: 3-7). In an aspect, a kit provided herein comprises one or more of the selected engineered analogs of ANP provided herein (e.g., one or more substantially pure selected engineered analogs of ANP that comprise, consist essentially of, or consist of the amino acid sequence set forth in any of SEQ ID NOs: 3-7) and nucleic acids encoding one or more selected engineered analogs of ANP provided herein (e.g., nucleic acid encoding one or more selected engineered analogs of ANP that comprise, consist essentially of, or consist of the amino acid sequence set forth in any of SEQ ID NOs: 3-7).

The examples set out herein illustrate one or more aspects of the present disclosure, but should not be construed as limiting the scope of the present disclosure in any manner.

### EXAMPLES

### Example 1: Synthesis of CRRL 191: Activator of GC-A

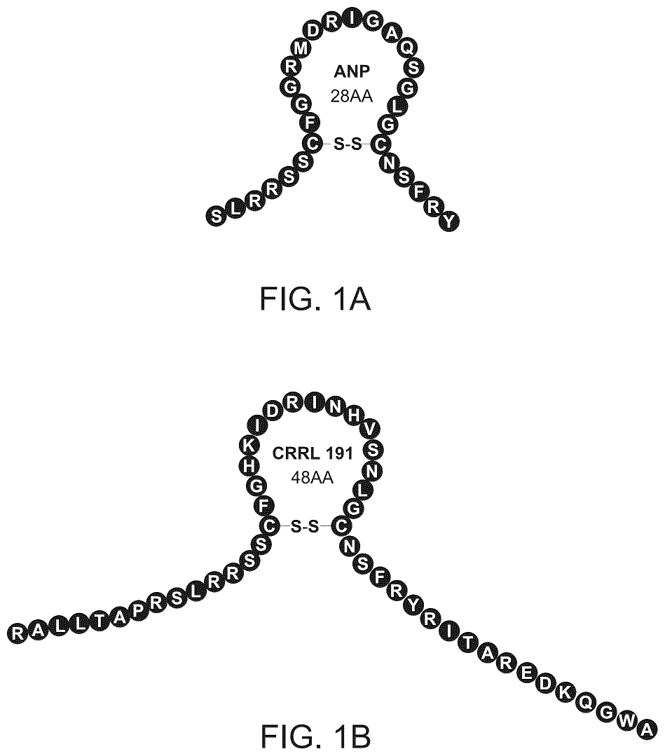

CRRL 191 is designed as a novel 48 amino acid (AA) designer natriuretic peptide which activates GC-A (FIG. 1B). CRRL 191 is synthesized by Bachem Ltd (Torrance, CA) using solid state synthetic peptide synthesis (see Table 2). After synthesis, the peptide is purified by high-performance liquid chromatography (HPLC), and is oxidized to form a cyclized ring between cysteine amino acid residues 15 and 31. The structure, as shown in FIG. 1B, is confirmed by MALDI mass spectrometry, and purity is measured to be 97.9% using HPLC analysis. The average mass of the cyclized CRRL 191 is 5557.27 Da.

TABLE 2
Analytical results from peptide synthesis

| Product | CRRL 191 acetate salt | |
|---|---|---|
| | H-Arg-Ala-Leu-Leu-Thr-Ala-Pro-Arg-Ser-Leu-Arg- | |
| | Arg-Ser-Ser-Cys-Phe-Gly-His-Lys-Ile-Asp-Arg-Ile- | |
| | Asn-His-Val-Ser-Asn-Leu-Gly-Cys-Asn-Ser-Phe-Arg- | |
| | Tyr-Arg-Ile-Thr-Ala-Arg-Glu-Asp-Lys-Gln-Gly-Trp- | |
| | Ala-OH acetate salt (Disulfide bond) | |
| Molecular | C₂₃₉H₃₈₅N₈₃O₆₇S₂ | |
| formula | | |
| Relative | 5557.27 | |
| molecular | | |
| mass | | |
| Appearance | White powder | |
| Identification | 5557.51 | |
| (MALDI-MS) | | |
| Amino acid | Asx 5.10 (5) | Ile 2.60 (3) |
| analysis | Thr 2.20 (2) | Leu 3.76 (4) |
| | Ser 4.79 (5) | Tyr 0.99 (1) |
| | Glx 2.10 (2) | Phe 1.97 (2) |
| | Pro* 1.51 (1) | His 1.85 (2) |
| | Gly 3.08 (3) | Lys 2.06 (2) |
| | Ala 4.05 (4) | Trp** 0.81 (1) |
| | Cys** 0.07 (2) | Arg 7.88 (8) |
| | Val 0.96 (1) | |

| | |
|---|---|
| | *Proline value high due to the incorporation of cysteine |
| | **Partially destroyed during acid hydrolysis |
| Solubility | Soluble in water at 1 mg/mL |
| Purity (HPLC) | 97.9% |
| Assay (AAA) | 77.6% |

### Example 2: Measurement of GC-A Activation and Cyclic GMP Production in HEK293 Cells

HEK293 cells stably transfected with human GC-A (cDNA clones from Origene, Rockville, MD) using Lipofectamine (Invitrogen, Grand Island, NY), are developed. Receptor overexpression is verified with immunofluorescence and western blotting. GC-A-transfected HEK293 (HEK/GCA) cells are maintained in Dulbecco's modified Eagle's medium (DMEM) supplemented with 10% fetal bovine serum (FBS), 100 U/ml penicillin, 100 U/ml streptomycin and 250 μg/ml G418.

Two in vitro cGMP assays are performed. Briefly, HEK/GCA cells are seeded in 48-well plates and cultured overnight to 80-90% confluency. The treatment buffer used in all experiments comprises Hank's Balanced Salt Solution (HBSS), 0.1% bovine serum albumin (BSA), 2 mM HEPES, and 0.5 mM 3-isobutyl-1-methylxanthine (IBMX, a non-specific phosphodiesterase inhibitor) (Sigma, St. Louis, MO).

In the first experiment, for test samples, cells are stimulated with ANP or CRRL191 at different concentrations (10⁻¹⁴ to 10⁻⁶M) for 10 minutes. After treatment, all cells are washed with phosphate buffered solution (PBS) once and lysed with 0.1M HCl. Intracellular cGMP is measured in the lysate using a commercial cGMP ELISA kit (Enzo Life Sciences, Farmingdale, NY) as instructed by the manufacturer.

As shown in Table 3, at 10 minutes of stimulation, the EC₅₀ demonstrated greater potency for CRRL191 compared to ANP.

TABLE 3
EC50 of CRRL191 or ANP in HEK293 GC-A Cells after 10 min stimulation.

| | ANP | | | | CRRL 191 | | | |
|---|---|---|---|---|---|---|---|---|
| Concentration | Mean | SD | EC₅₀ | Cₘₐₓ | Mean | SD | EC₅₀ | Cₘₐₓ |
| 10⁻¹⁴ M | 3.49 | 0.72 | 7.95 × 10⁻¹⁰ M | 143.9 | 3.07 | 0.24 | 4.8 × 10⁻¹⁰ M | 143.3 |
| 10⁻¹² M | 4.68 | 0.65 | | | 2.57 | 0.14 | | |
| 10⁻¹⁰ M | 26.35 | 4.27 | | | 35.57 | 1.61 | | |
| 10⁻⁹ M | 79.87 | 13.55 | | | 89.94 | 3.01 | | |
| 10⁻⁸ M | 138.05 | 12.89 | | | 152.04 | 5.83 | | |
| 10⁻⁷ M | 127.08 | 18.45 | | | 140.84 | 0.44 | | |
| 10⁻⁶ M | 157.60 | 14.01 | | | 135.48 | 6.89 | | |

In the second experiment, ANP or CRRL 191 (at 10⁻⁸ M) are added to the treatment buffer and the cells are incubated for 6 hours. After treatment, all cells are washed with phosphate buffered solution (PBS) once and lysed with 0.1M HCl. Intracellular cGMP is measured in the lysate using a commercial cGMP ELISA kit (Enzo Life Sciences, Farmingdale, NY) as instructed by the manufacturer.

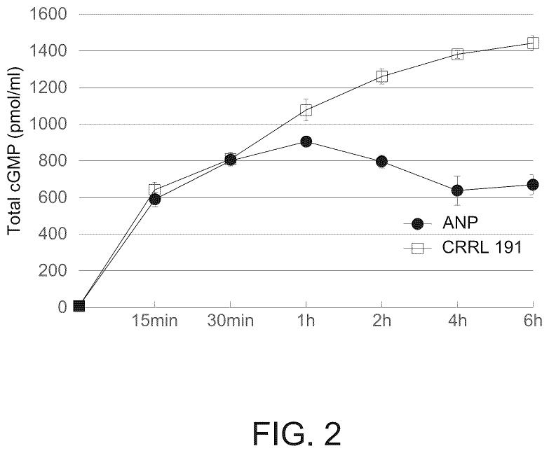

As shown in FIG. 2, both ANP and CRRL 191 activate GC-A receptors with increases in cGMP production at the same rate up to about 30 minutes. After 30 minutes, GC-A receptor activation by CRRL 191 continues to increase up to at least 6 hours, as demonstrated by increasing cGMP production, while GC-A receptor activation by ANP plateaus and decreases.

### Example 3: In Vitro Neprilysin Degradation Assay

In vitro degradation of ANP and CRRL 191 peptides by recombinant human neprilysin (NEP) (R&D systems, Minneapolis, MN) is measured by cGMP production in HEK/GCA cells. Briefly, 5 μl of 2×10⁻⁵ M peptide is incubated with 5 μl (10 ng/μl) recombinant NEP in 90 μl Tris/0.1% BSA buffer (50 mM Tris-HCl, pH 7.5, 150 mM NaCl, 5 mM MgCl₂, 0.1% BSA) at 37° C. for various lengths of time ranging from 15 min to 24 hours. Peptides for the 0-min timepoint are incubated without NEP (buffer only). After incubation for the required length of time (0-min to 24-hour), 100 μl of 0.5 N perchloric acid is added to the reaction solution to inactivate NEP and stop degradation, and 20 μl of 2.5 N NaOH is added to neutralize the solution. The neutralized reaction aliquot is added to HEK/GCA cells and the residual capability of the undegraded peptide (diluted to a final concentration 5.5×10⁻⁸ M) to produce cGMP is measured using the same commercial cGMP ELISA kit (Enzo Life Sciences, Farmingdale, NY) as described in Example 2, following the instructions of the manufacturer. Degradation of ANP and CRRL 191 is calculated by the loss of cGMP-generating capability in HEK/GCA cells compared to the basal cGMP-generating capability at 0 min.

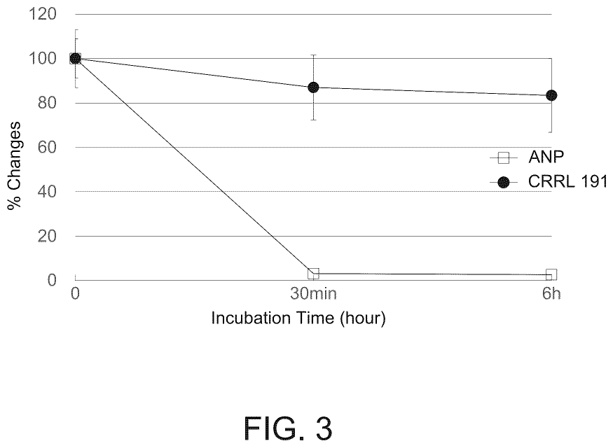

As shown in FIG. 3, CRRL 191 is highly resistant to degradation by neprilysin compared to ANP as measured by this in vitro assay. After 30 minutes of neprilysin degradation, CRRL 191 retains about 90% of its original bioactivity, while ANP retains less than 5% of its original bioactivity. After 6 hours of neprilysin degradation, CRRL 191 retains 84% of its original bioactivity, demonstrating marked resistance and stability compared to ANP.

### Example 4: Human Cardiomyocyte (HCM) cGMP Analysis

Human cardiomyocytes (HCM) are purchased from ScienCell (Catalog #6200) and maintained in Cardiac Myocyte Medium (Cat #6101, ScienCell) containing 10% FBS. An in vitro cGMP assay is performed. Briefly, HCM cells are seeded in 6-well plates and cultured overnight to 80-90% confluency. The treatment buffer used in all experiments comprises Hank's Balanced Salt Solution (HBSS), 0.1% BSA, 2 mM HEPES, and 0.5 mM 3-isobutyl-1-methylxanthine (IBMX, a non-specific phosphodiesterase inhibitor) (Sigma, St. Louis, MO). For test samples, HCM are incubated for 10 minutes in treatment buffer containing ANP or CRRL 191 (at 10⁻¹² M, 10⁻¹¹ M, 10⁻¹⁰ M, 10⁻⁹ M, 10⁻⁸ M, 10⁻⁷ M, and 10⁻⁶ M). For the negative control, HCM are incubated in treatment buffer (vehicle) only. After treatment for 10 minutes, all cells are washed with phosphate buffered solution (PBS) once, lysed with 0.1 M HCl, and sonicated for 10 min. Intracellular cGMP is measured in the lysate using a commercial cGMP ELISA kit (Enzo Life Sciences, Farmingdale, NY) as instructed by the manufacturer.

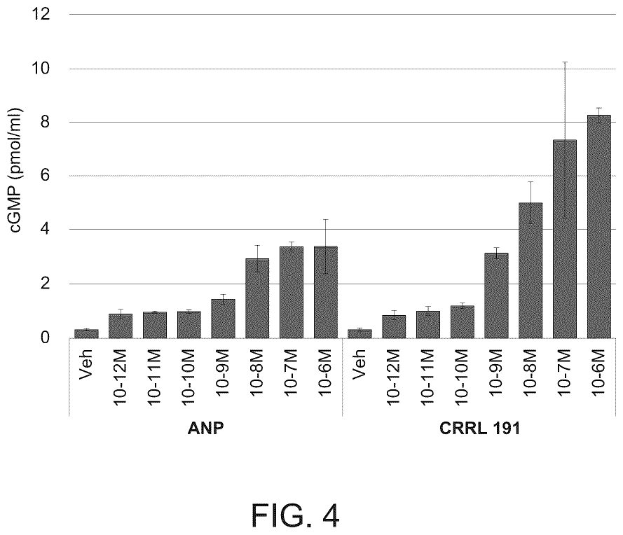

As shown in FIG. 4, CRRL 191 induces greater levels of cGMP production in human primary cells. From 10⁻¹²M to 10⁻¹¹ M, CRRL 191 and ANP induce similar levels of cGMP production. From 10⁻¹⁰ M to 10⁻⁶ M, CRRL 191 induces greater cGMP production than ANP.

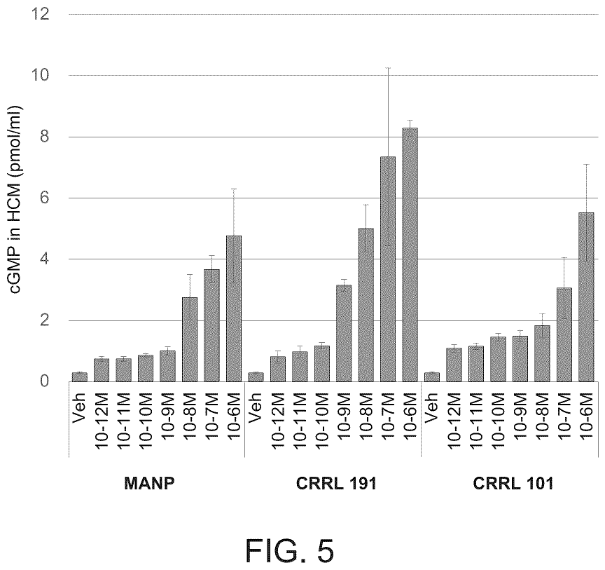

As shown in FIG. 5, CRRL 191 induces greater levels of cGMP production in human primary cells compared to two other GC-A activators, MANP and CRRL 101. From 10⁻⁹ M to 10⁻⁶ M, CRRL 191 induced great levels of cGMP production than MANP or CRRL 101.

### Example 5: Human Cardiomyocyte (HCM) Real-time Apoptosis Analysis

HCM cells are seeded in 96-well plate at approximately 80% confluency. Cells are treated with various concentrations of staurosporine (SP) to induce apoptosis. Based on the dose response curve, 0.05 μM of SP is selected to induce apoptosis in HCM.

On the day of the experiment, HCM are incubated with cell growth medium containing 0.05 μM of SP, various concentration of ANP or CRRL191, and 5 pmol/L IncuCyte Caspase-3/7 Green Reagent (Essen Bioscience, Ann Arbor, MI). Plates are then transferred to a time-lapsed, live imaging system IncuCyte S3 (Essen BioScience, Ann Arbor, MI) for apoptosis monitoring every 2 hours for a total of 90 hours. All treatment groups are performed in quintuplicates and repeated twice. Images are collected using the IncuCyte S3 Live-Cell Analysis System Software (Essen Bioscience, Ann Arbor, MI), and the data are analyzed for the Green Object Area of apoptotic bodies per well.

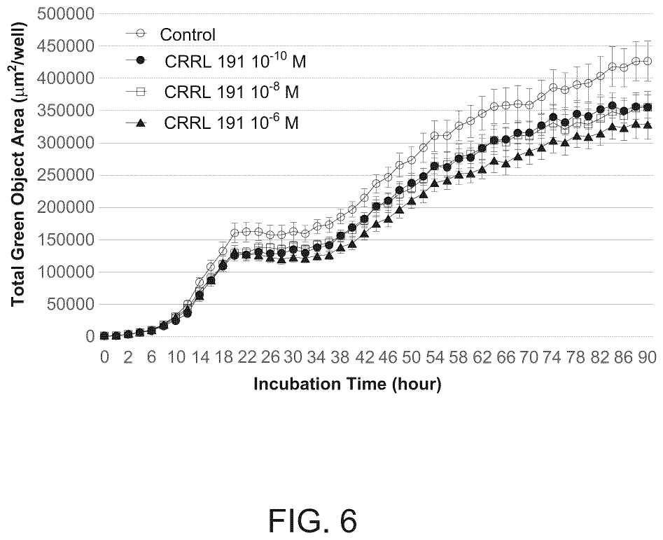

As shown in FIG. 6, CRRL 191 at doses of 10⁻¹⁰ and 10⁻⁸M reduced cardiomyocyte apoptosis induced by SP, while CRRL 191 at a dose 10⁻⁶M had the greatest effect on reducing cardiomyocyte apoptosis induced by SP.

### Example 6: GC-A Binding Studies

Surface plasmon resonance (SPR) measurements are performed at 25° C. on a BI-4500 SPR instrument (Biosensing Instrument Inc., Tempe, AZ). The extracellular domain of GC-A recombinant protein with a C-terminal His-tag (MyBioSource, Inc., San Diego, CA) is immobilized onto a Ni-NTA sensor chip (Biosensing Instrument Inc., Tempe, AZ) using 400 μM nickel sulfate in de-iron water as linker, as instructed by the instrument manual. Then, 40 μg/ml of recombinant GC-A is immobilized on the Ni-NTA sensor chip. After the chip is washed with buffer (150 mM NaCl, 50 μM EDTA, pH 7.4, 0.1% DMSO), 100 μL of serially diluted CRRL 191 or ANP (0.125 nM, 0.25 nM, 0.5 nM, 1 nM, 2 nM) is injected at the rate of 60 μL/min and allowed to dissociate for 60 seconds. Data are collected as sensorgrams. Binding kinetics are calculated from sensorgrams using the BI-Data Analysis Program (Biosensing Instrument, Tempe, AZ).

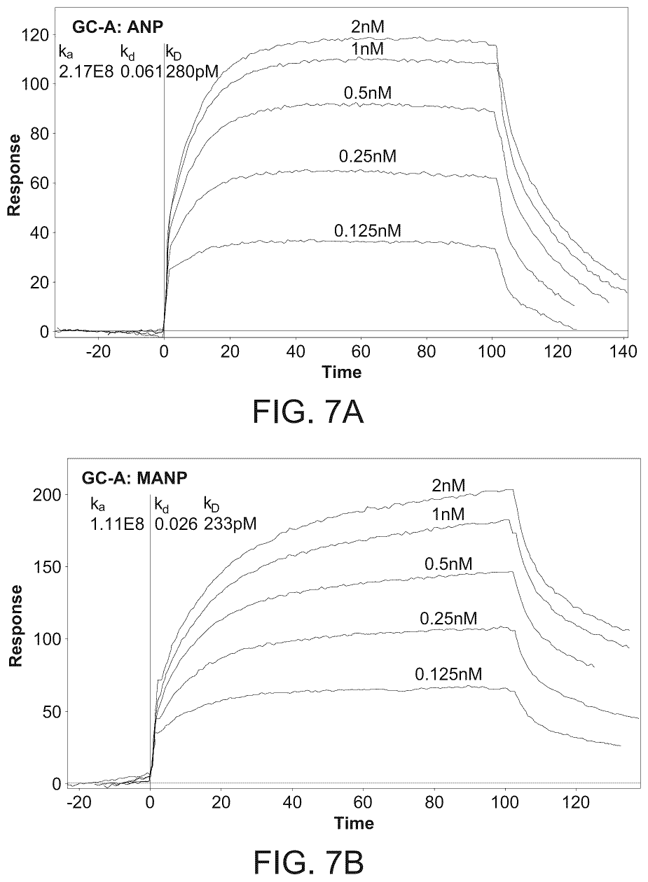

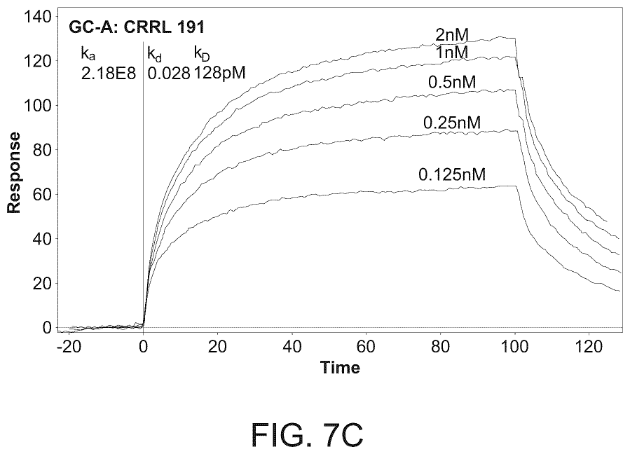

As shown in the binding curves, CRRL 191 (FIG. 7C) shows greater binding for the GC-A receptor than ANP (FIG. 7A) and MANP (FIG. 7B). The K_{D} value for CRRL 191 is 128 μM, while the K_{D} value for ANP and MANP are 280 μM and 233 μM respectively. These data demonstrate that CRRL 191 has superior binding to human GC-A than ANP or MANP.

### Example 7: In Vivo Rat Experiments

All rat experiments are conducted following the Animal Welfare Act and the protocol is approved by the Mayo Clinic Institutional Animal Care and Use Committee (IACUC). A total of 19 Spontaneously Hypertensive Rats (SHR, male, 250-300 g) are randomly assigned to infusion with one of the following: (i) vehicle (0.9% saline, n=3), (ii) low dose ANP (100 pmol/kg/min, n=4), (iii) high dose ANP (300 pmol/kg/min, n=4), (iv) low dose CRRL 191 (100 pmol/kg/min, n=4), and (v) high dose CRRL 191 (300 pmol/kg/min, n=4). Rats are included only if they are deemed healthy by the Institutional Veterinarian Department. Investigators who conduct the in vivo study are not blinded to treatments, but biochemical analysis is performed by a different investigator who is blinded to treatment.

On the day of the experiment, SHR rats are anesthetized with inactin (120 mg/kg, intraperitoneal injection, Sigma, St. Louis, MO). Rats are then kept on a heating pad at 38° C. to maintain a normal body temperature for the entire study. Vascular and bladder cannulation procedures are conducted. Briefly, a polyethylene-50 (PE-50) tube catheter is placed into one jugular vein for peptide infusion and another PE-50 tube is placed into the carotid artery for blood pressure (BP) monitoring (Sonometrics, London, Ontario, Canada) and blood sampling. After instrumentation, a 15-minute equilibrium period follows, and a 30-minute pre-infusion period is performed to collect baseline urine samples. The pre-infusion period is followed by a 45-minute continuous infusion of CRRL 191, ANP, or vehicle (15-minute lead-in drug infusion, 30-minute clearance during drug infusion).

Immediately after peptide infusion is stopped, another 30-minute washout clearance period is started before termination and sacrifice. Four blood samples are collected to determine circulating cGMP levels: at baseline, before the end of drug infusion, at the middle of the washout clearance period, and before the end of the washout clearance period. Any blood loss in the rats is replaced with an equal volume of saline. BP is monitored and recorded every 15 minutes beginning after the initiation of peptide infusion. All collected blood are stored at −80° C. until assayed.

For the biochemical analysis, plasma cGMP levels are measured with a cGMP ELISA kit (Enzo Life Sciences, Farmingdale, NY).

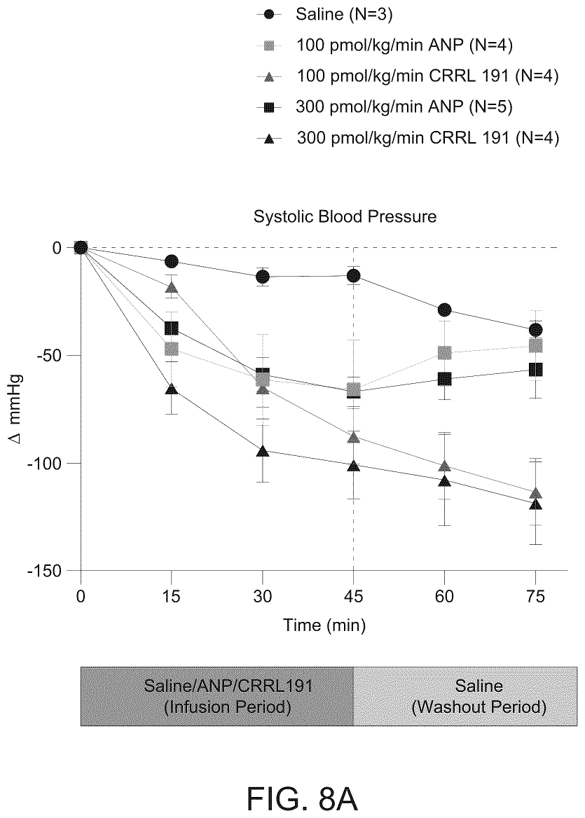

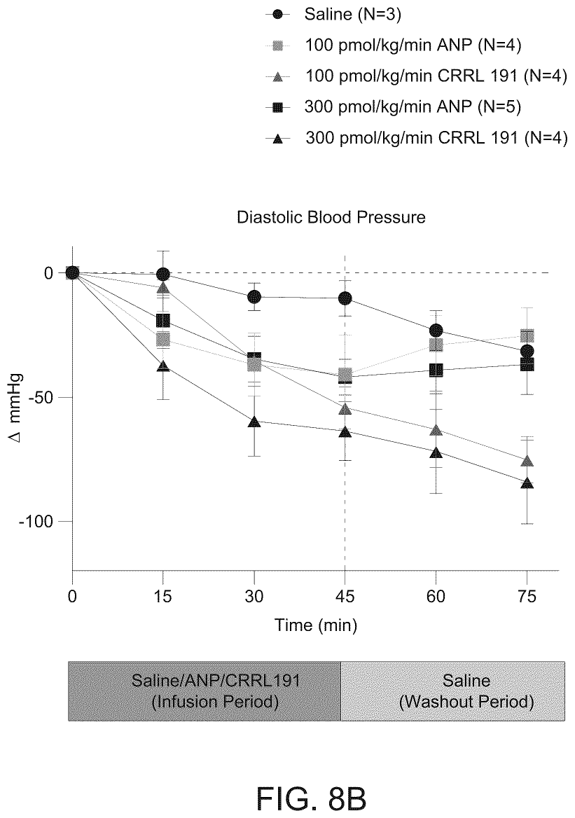

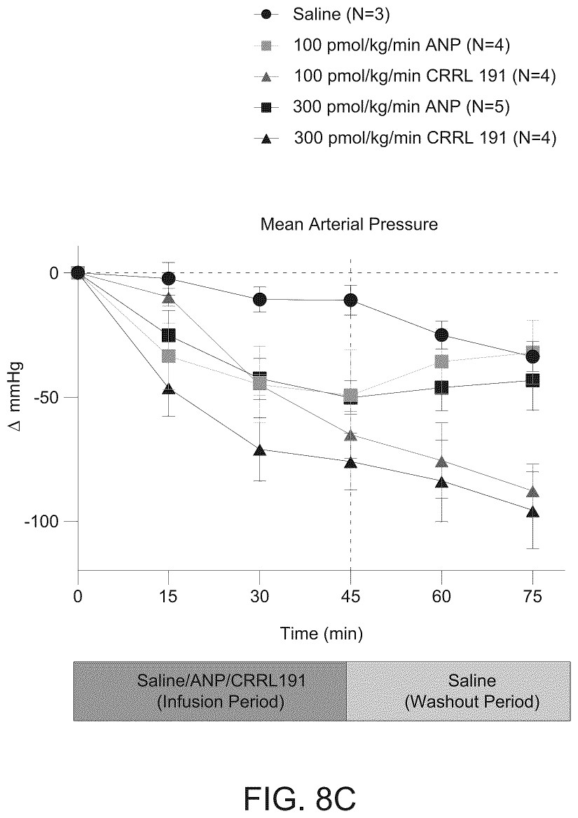

CRRL 191 is a more effective anti-hypertensive compared to ANP and vehicle. As shown, both 100 pmol/kg and 300 pmol/kg doses of CRRL 191 are more effective at lowering systolic (FIG. 8A), diastolic (FIG. 8B), and mean arterial blood pressure (FIG. 8C) in spontaneously hypertensive rats than 100 pmol/kg or 300 pmol/kg of ANP, with a more sustained effect over time after peptide administration.

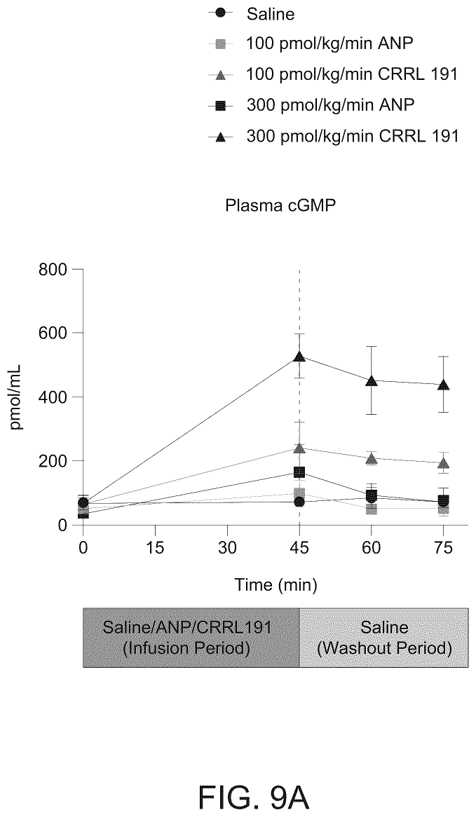

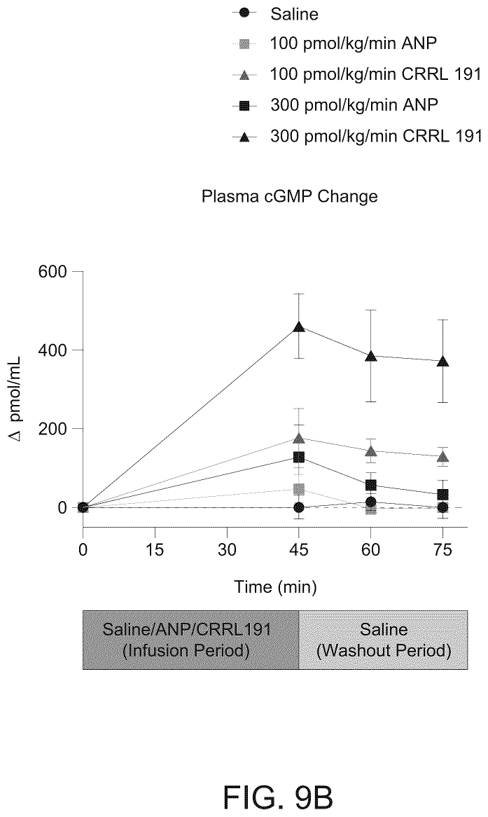

Finally, plasma cGMP levels increase in spontaneously hypertensive rats when given ANP or CRRL 191 (FIG. 9A). However, both 100 pmol/kg and 300 pmol/kg doses of CRRL 191 are more potent compared to 100 pmol/kg or 300 pmol/kg of ANP in activating cGMP in plasma, with a more sustained activation after peptide administration (FIG. 9B).

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims. A list of exemplary embodiments is provided:
Embodiment 1. A polypeptide comprising, consisting essentially of, or consisting of the amino acid sequence set forth in any one of SEQ ID NOs: 3-7.
Embodiment 2. A polypeptide comprising, consisting essentially of, or consisting of the amino acid sequence set forth in any one of SEQ ID NOs: 3-7, wherein the polypeptide further comprises one, two, or three amino acid substitutions, additions, or deletions.
Embodiment 3. A polypeptide comprising, consisting essentially of, or consisting of a sequence having at least 90% homology with any one of SEQ ID NO: 3-7.
Embodiment 4. The polypeptide of Embodiment 3, wherein the sequence has at least 95% homology with any one of SEQ ID NO: 3-7.
Embodiment 5. The polypeptide of Embodiment 3, wherein the sequence has at least 97% homology with any one of SEQ ID NO: 3-7.
Embodiment 6. A polypeptide comprising, consisting essentially of, or consisting of the amino acid sequence set forth in amino acids 1 to 8 of SEQ ID NO 7.
Embodiment 7. A substantially pure polypeptide comprising, consisting essentially of, or consisting of an amino acid sequence set forth in any one of SEQ ID NOs: 3-7.
Embodiment 8. A substantially pure polypeptide comprising, consisting essentially of, or consisting of an amino acid sequence having at least 90% homology with any one of SEQ ID NOs: 3-7.
Embodiment 9. The substantially pure polypeptide of Embodiment 8, wherein the sequence has at least 95% homology with any one of SEQ ID NO: 3-7.
Embodiment 10. The substantially pure polypeptide of Embodiment 8, wherein the sequence has at least 97% homology with any one of SEQ ID NO: 3-7.
Embodiment 11. A substantially pure polypeptide comprising, consisting essentially of, or consisting of the amino acid sequence set forth in any one of SEQ ID NOs: 3-7, wherein the polypeptide comprises one, two, or three amino acid substitutions, additions, or deletions.
Embodiment 12. A substantially pure polypeptide comprising, consisting essentially of, or consisting of the amino acid sequence set forth in amino acids 1 to 8 of SEQ ID NO 7.
Embodiment 13. A composition comprising a polypeptide comprising the amino acid sequence set forth in any one of SEQ ID NOs: 3-7.
Embodiment 14. A composition comprising a polypeptide comprising, consisting essentially of, or consisting of the amino acid sequence set forth in any one of SEQ ID NOs: 3-7, wherein the polypeptide further comprises one, two, or three amino acid substitutions, additions, or deletions.
Embodiment 15. A composition comprising at least two polypeptides, wherein each of said at least two polypeptides is a polypeptide comprising, consisting essentially of, or consisting of the amino acid sequence set forth in any one of SEQ ID NOs: 3-7.
Embodiment 16. A composition comprising, consisting essentially of, or consisting of the amino acid sequence set forth in amino acids 1 to 8 of SEQ ID NO 7.
Embodiment 17. The composition of any one of Embodiments 13-16, further comprising one or more pharmaceutically-acceptable excipient.
Embodiment 18. The composition of any one of Embodiments 13-17, further comprising an agent selected from the group consisting of a diuretic, an angiotensin-converting enzyme (ACE) inhibitor, an angiotensin II receptor blocker (ARB), and a calcium channel blocker (CCB).
Embodiment 19. The composition of any one of Embodiments 13-18, further comprising furosemide.
Embodiment 20. A nucleic acid encoding a polypeptide comprising, consisting essentially of, or consisting of the amino acid sequence set forth in any one of SEQ ID NOs: 3-7.
Embodiment 21. A composition comprising nucleic acid encoding a polypeptide comprising, consisting essentially of, or consisting of the amino acid sequence set forth in any one of SEQ ID NOs: 3-7.
Embodiment 22. A composition comprising nucleic acid encoding at least two polypeptides, wherein each of said at least two polypeptides is a polypeptide comprising, consisting essentially of, or consisting of the amino acid sequence set forth in any one of SEQ ID NOs: 3-7.
Embodiment 23. The composition of any one of Embodiments 21-22, further comprising an agent selected from the group consisting of a diuretic, an angiotensin-converting enzyme (ACE) inhibitor, an angiotensin II receptor blocker (ARB), and a calcium channel blocker (CCB).
Embodiment 24. The composition of any one of Embodiments 21-23, further comprising furosemide.
Embodiment 25. The composition of any one of Embodiments 21-24, wherein said nucleic acid is in the form of a non-viral vector.
Embodiment 26. The composition of Embodiment 25, wherein said non-viral vector is an expression plasmid.
Embodiment 27. The composition of any one of Embodiments 21-24, wherein said nucleic acid is in the form of a viral vector.
Embodiment 28. A method for treating a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) in a mammal, wherein said method comprises administering to said mammal a composition comprising a polypeptide comprising, consisting essentially of, or consisting of the amino acid sequence set forth in any one of SEQ ID NOs: 3-7 or nucleic acid encoding said polypeptide.
Embodiment 29. The method of Embodiment 28, wherein said mammal is a human.
Embodiment 30. The method of any one of Embodiments 28-29, wherein said cardiovascular, cardiorenal, or metabolic disease is hypertension.
Embodiment 31. The method of any one of Embodiments 28-30, wherein said cardiovascular, cardiorenal, or metabolic disease is resistant hypertension.
Embodiment 32. The method of any one of Embodiments 28-31, wherein said composition comprises an agent selected from the group consisting of a diuretic, an angiotensin-converting enzyme (ACE) inhibitor, an angiotensin II receptor blocker (ARB), and a calcium channel blocker (CCB).
Embodiment 33. The method of any one of claims Embodiments 28-32, wherein said composition comprises furosemide.
Embodiment 34. A method for treating a mammal at risk of developing a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH), wherein said method comprises administering to said mammal a composition comprising a polypeptide comprising, consisting essentially of, or consisting of the amino acid sequence set forth in any one of SEQ ID NOs: 3-7 or nucleic acid encoding said polypeptide.
Embodiment 35. The method of Embodiment 34, wherein said mammal is a human.
Embodiment 36. The method of any one of Embodiments 34-35, wherein said cardiovascular, cardiorenal, or metabolic disease is hypertension.
Embodiment 37. The method of any one of Embodiments 34-36, wherein said cardiovascular, cardiorenal, or metabolic disease is resistant hypertension.
Embodiment 38. The method of any one of Embodiments 34-37, wherein said composition comprises an agent selected from the group consisting of a diuretic, an angiotensin-converting enzyme (ACE) inhibitor, an angiotensin II receptor blocker (ARB), and a calcium channel blocker (CCB).
Embodiment 39. The method of any one of Embodiments 34-38, wherein said composition comprises furosemide.
Embodiment 40. A method for alleviating the symptoms of a cardiovascular (e.g., hypertension, heart failure, cardiomyopathies, valvular disease, myocardial infarction) cardiorenal (e.g., acute kidney injury, chronic kidney disease, polycystic kidney disease, chemotherapeutic-induced cardiac and renal dysfunction), or metabolic disease (e.g., diabetes, obesity, metabolic syndrome, hyperlipidemia, insulin resistance, primary aldosteronism, NASH) in a mammal, wherein said method comprises administering to said mammal a composition comprising a polypeptide comprising, consisting essentially of, or consisting of the amino acid sequence set forth in any one of SEQ ID NOs: 3-7 or nucleic acid encoding said polypeptide.
Embodiment 41. The method of Embodiments 40, wherein said mammal is a human.
Embodiment 42. The method of any one of Embodiments 40-41, wherein said cardiovascular, cardiorenal, or metabolic disease is hypertension.
Embodiment 43. The method of any one of Embodiments 40-42, wherein said cardiovascular, cardiorenal, or metabolic disease is resistant hypertension.
Embodiment 44. The method of any one of Embodiments 40-43, wherein said composition comprises an agent selected from the group consisting of a diuretic, an angiotensin-converting enzyme (ACE) inhibitor, an angiotensin II receptor blocker (ARB), and a calcium channel blocker (CCB).
Embodiment 45. The method of any one of Embodiments 40-44, wherein said composition comprises furosemide.

1. (canceled)

2. (canceled)

3. A polypeptide comprising a sequence having at least 90% homology with any one of SEQ ID NO: 3-7.

4. The polypeptide of claim 3, wherein the sequence comprises at least one modification selected from the group consisting of: an amino acid substitution, an amino acid addition, and an amino acid deletion.

5. The polypeptide of claim 3, wherein the sequence comprises the amino acid sequence set forth in any one of SEQ ID NOs: 3-7.

6.-12. (canceled)

13. A composition comprising a polypeptide comprising an amino acid sequence having at least 90% homology with any one of SEQ ID NOs: 3-7.

14.-16. (canceled)

17. The composition of claim 13, further comprising one or more pharmaceutically-acceptable excipient.

18. The composition of claim 13, further comprising an agent selected from the group consisting of a diuretic, an angiotensin-converting enzyme (ACE) inhibitor, an angiotensin II receptor blocker (ARB), and a calcium channel blocker (CCB).

19. (canceled)

20. A nucleic acid encoding a polypeptide comprising an amino acid sequence having at least 90% homology with any one of SEQ ID NOs: 3-7.

21.-27. (canceled)

28. A method for treating or preventing a cardiovascular cardiorenal, or metabolic disease in a mammal, wherein said method comprises administering to said mammal a composition comprising (i) a polypeptide comprising an amino acid sequence having at least 90% homology with any one of SEQ ID NOs: 3-7, (ii) a polypeptide comprising amino acids 1 to 8 of SEQ ID NO: 7, (iii) a nucleic acid encoding a polypeptide comprising amino acids 1 to 8 of SEQ ID NO: 7, or iv a nucleic acid encoding a polypeptide comprising an amino acid sequence having at least 90% homology with any one of SEQ ID NOs: 3-7.

29. The method of claim 28, wherein said mammal is a human.

30. The method of claim 28, wherein said cardiovascular, cardiorenal, or metabolic disease is hypertension.

31. The method of claim 28, wherein said cardiovascular, cardiorenal, or metabolic disease is resistant hypertension.

32. The method of claim 28, wherein said composition comprises an agent selected from the group consisting of a diuretic, an angiotensin-converting enzyme (ACE) inhibitor, an angiotensin II receptor blocker (ARB), and a calcium channel blocker (CCB).

33. The method of claim 28, wherein said mammal is at risk of developing a cardiovascular, cardiorenal, or metabolic disease.

34-45. (canceled)


SEQUENCE LISTING (7 sequences)

SEQ ID NO: 1 - protein, 28 aa, synthetic construct
SLRRSSCFGGRMDRIGAQSGLGCNSFRY

SEQ ID NO: 2 - protein, 40 aa, synthetic construct
SLRRSSCFGGRMDRIGAQSGLGCNSFRYRITAREDKQGWA

SEQ ID NO: 3 - protein, 40 aa, synthetic construct
SLRRSSCFGHKIDRINHVSNLGCNSFRYRITAREDKQGWA

SEQ ID NO: 4 - protein, 41 aa, synthetic construct
SLRRSSCFGHKIDRINHVSSNLGCNSFRYRITAREDKQGWA

SEQ ID NO: 5 - protein, 41 aa, synthetic construct
SLRRSSCFGHKIDRINHVSNNLGCNSFRYRITAREDKQGWA

SEQ ID NO: 6 - protein, 44 aa, synthetic construct
TAPRSLRRSSCFGHKIDRINHVSNLGCNSFRYRITAREDKQGWA

SEQ ID NO: 7 - protein, 48 aa, synthetic construct
RALLTAPRSLRRSSCFGHKIDRINHVSNLGCNSFRYRITAREDKQGWA